(19) United States
(12) Patent Application Publication
(10) Pub. No.: US 2022/0034879 A1
(43) Pub. Date: Feb. 3, 2022

(54) MEANS AND METHOD FOR POINT-OF-CARE ANALYSIS OF LIQUID SAMPLES

(71) Applicant: The State of Israel, Ministry of Agriculture & Rural Development, Agricultural Research Organization, Rishon Lezion (IL)

(72) Inventor: Evgeni ELZTOV, Ashdod (IL)

(21) Appl. No.: 17/288,542

(22) PCT Filed: Oct. 24, 2019
(86) PCT No.: PCT/IL2019/051150

§ 371 Date: Apr. 25, 2021

Related U.S. Application Data
(60) Provisional application No. 62/750,260, filed on Oct. 25, 2018.

(51) Int. Cl. G01N 33/558; G01N 33/52; G01N 33/543

(52) U.S. Cl. CPC G01N 33/558; G01N 33/569; G01N 33/543; G01N 33/52

(57) ABSTRACT

A sensor for the rapid, onsite identification of analytes characterized by: (a) a sample layer; (b) at least one reaction layer, interconnected with the sample layer; (c) at least one reporter layer, interconnected with the reaction payer; (d) at least one preventative layer, interconnected with the reporter layer; and (e) an absorption pad, interconnected with the preventative layer; with the layers arranged as a signaling channel.

### FIELD OF THE INVENTION

The field of the invention is system for point-of-care analysis of samples.

### BACKGROUND

The infestation or contamination by pathogens, pollutions or toxins in water or food is a major cause of disease and sickness around the world. This is a particular problem in in the developing world where there is a lack of technology and budget for tracking the contamination of water sources and of the food chain. The WHO estimates that 1.5 million deaths a year are caused by waterborne diseases, over half due to safe water supply, sanitation and hygiene (WHO: Burden of disease and cost-effectiveness estimates, 2014), while foodborne illness was responsible for 420,000 deaths in 2010 (WHO estimates of the global burden of foodborne diseases, 2015).

Current technologies, such as GS-MS, Mass spectrometry or HPLC are expensive, immobile and need highly trained operators.

The present invention proposes a point-of-care sensor that provides a simple, portable and cost efficient solution, and to provide rapid and localized analysis of markers, pathogens, pollutions, toxins or vectors for the outbreak of infectious diseases. Biomarker can be characterized as proteins, metabolites, antibodies, peptides, hormones, lipids etc in addition to whole cells. Alternatively the analytes could be fragments of the original biomarker created by fragmentation, desintegration, deterioration, decay etc.

There exists a number of methods identify pathogens pollutions or toxins, such as cell plate culture, immunoassays, and nucleic acid related tests. However, these methods have various disadvantages such as low sensitivity, high price, assay complexity, requirement of a lab environment, and more. The adaptation of these methods for field use has proven to be challenging.

Two types of devices have reached the necessary requirements to enable wide spread consumer use. One is the biosensor-based glucometer, and the other is the pregnancy test.

Lateral flow immunoassay (LFA) is currently the technology with the most potential to meet the challenge.

A number of advances have been made in developing cost-effective and rapid bacterial testing based on the lateral flow technology, with sensors for Escherichia coli, Listeria, Salmonella and Streptococcus being developed. However, low sensitivity and low specificity to the target analyte limit their use. ELISA based technologies (e.g. chemo-luminescence, electrochemistry and, colorimetry) provide higher sensitivity and specificity, however they are more complicated and time consuming.

WO201897796 describes a device to determine or quantify the presence of an analyte molecule, virus or cell of interest in a sample. However '796 contains the additional steps of preparing a conjugated analyte-reporter molecule.

U.S. Pat. No. 7,300,802B2 describes a biosensor for point-of-care testing (POCT) whose detection sensitivity is improved by introducing successive cross-flow procedure for immune reaction and enzymatic reaction to a membrane strip chromatographic assay system. However the biosensor described in '802 must be operated by a skilled professional due to the use of multiple systems.

Therefore their exists an unmet need for a rapid, onsite, simple and low-cost system for the detection of contaminated +food and water sources+.

### SUMMARY

It is an object of the present invention to provide a sensor for the rapid, onsite identification of analytes characterized by:
a. a sample layer;
b. at least one reaction layer, interconnected with the sample layer:
c. at least one reporter layer, interconnected with the reaction payer;
d. at least one preventative layer, interconnected with the reporter layer; and
e. an absorption pad, interconnected with the preventative layer;
   wherein the layers are arranged from a-to-e and are operatively arranged as a specific analyte signaling channel.

It is another object of the present invention to provide the aforementioned sensor, comprising
a. a sample layer;
b. at least one reporter layer, interconnected with the sample layer;
c. at least one reaction layer, interconnected with the reporter layer;
d. at least one preventative layer, interconnected with the reaction layer; and
e. an absorption layer, interconnected with the preventative layer;
wherein the layers are arranged from a-to-e and are operatively arranged as a specific analyte signaling channel.

It is another object of the present invention to provide the aforementioned, wherein the sample layer is constructed from a porous, absorbent and nonreactive membrane.

It is another object of the present invention to provide the aforementioned sensor, wherein the membrane is selected from a group comprising: cellulose acetate membrane, nitrocellulose membrane, cellulose ester membrane, polysulfone (PS) membrane, polyether sulfone (PES) membrane, polyacrylonitrile (PAN) membrane, polyamide membrane, polyimide membrane, polyethylene and polypropylene (PE and PP) membrane, polytetrafluoroethylene (PTFE) membrane, polyvinylidene fluoride (PVDF) membrane, polyvinylchloride (PVC) membrane and fiberglass paper membrane.

It is another object of the present invention to provide the aforementioned sensor, wherein the reaction pad contains at least one kind of immobilized analyte-effector complex.

It is another object of the present invention to provide the aforementioned sensor, wherein the analytic-effector complex comprising of:
a. at least one analyte; and
b. at least one effector, the effector specific to the composition of the preventative pad;
wherein the analyte and effector are reversibly or irreversibly connected.

It is another object of the present invention to provide the aforementioned sensor, wherein the immobilized analyte-effector complex is reversibly bound to a specific anti-analyte antibody, the antibody is specific for the analyte.

It is another object of the present invention to provide the aforementioned sensor, wherein the analyte-effector complex is released from the antibody through specific competitive or non-competitive binding of the free analyte from the sample to the anti-body, immobilized to the reaction layer.

It is another object of the present invention to provide the aforementioned sensor, wherein the immobilized analyte-effector complex additionally comprises at least one reporter compound.

It is another object of the present invention to provide the aforementioned sensor, wherein the anti-analyte antibody is irreversibly bound to the reaction pad.

It is another object of the present invention to provide the aforementioned sensor, wherein the reporter layer is loaded with bound or unbound reporter compounds.

It is another object of the present invention to provide the aforementioned sensor, wherein the reporter compound characterized by being:
a. incapable of crossing through undigested, whole preventative layer;
b. capable of crossing through pores created in preventative layer; and
c. specific to the absorption pad or the detector.

It is another object of the present invention to provide the aforementioned sensor, wherein the reporter compound is selected from a group of compounds consisting of: dyes, pigments, electrochemical active compounds, enzymes, flourophores, chemiluminescent molecules and radionuclides.

It is another object of the present invention to provide the aforementioned sensor, wherein the reporter compound is blocked from crossing the unaffected preventative layer due to size, electric or magnetic charge, hydrophobicity or lipophobicity.

It is another object of the present invention to provide the aforementioned sensor, wherein preventative layer consists of at least one compound that is affected by the effector.

It is another object of the present invention to provide the aforementioned sensor, wherein the preventative layer is comprised of material that can be digested by an enzyme, the material selected from a group, the group consisting of sugars, hydrogels, peptides, lipids and polymers.

It is another object of the present invention to provide the aforementioned sensor, wherein the absorption pad selectively or non-selectively reacts or binds with the reporter compound to generate a signal.

It is another object of the present invention to provide the aforementioned sensor, wherein the signal is identified visually or measured by a detector.

It is another object of the present invention to provide the aforementioned sensor, wherein the detector is based on spectroscopic, photochemical, biochemical, enzymatic, immunochemical, electrical, radiographic and optical means.

It is another object of the present invention to provide the aforementioned sensor, wherein the detector communicates results to a computer system or network.

It is an object of the present invention to provide a sensor for the rapid onsite identification of analytes comprising:
a. at least one reversibly immobilized analyte-effector complex;
b. at least one reporter compound;
c. at least one preventative layer; and
d. at least one absorption layer;
   wherein the reporter compound and the analyte are not bound to each other; wherein the preventative layer is positioned between the analyte and the reporter and between the absorption layer and is configures as a specific analyte signaling channel.

It is another object of the present invention to provide the aforementioned sensor, wherein the analyte is bound to an effector to create an analyte-effector complex.

It is another object of the present invention to provide the aforementioned sensor, wherein the effector is an enzyme.

It is another object of the present invention to provide the aforementioned sensor, wherein the analyte-effector complex is immobilized by being reversibly bound to an anti-analyte antibody, the anti-analyte antibody is specific for the analyte.

It is another object of the present invention to provide the aforementioned sensor, wherein the analyte-effector complex is released from the antibody through specific competitive or non-competitive binding of the free analyte from the sample to the anti-body, immobilized to the reaction layer.

It is another object of the present invention to provide the aforementioned sensor, wherein the reporter compound is characterized by being:
a. incapable of flowing/traversing/passing/crossing through un-affected preventative layer; and
b. capable of flowing/traversing/passing/crossing through affected preventative layer.

It is another object of the present invention to provide the aforementioned sensor, wherein the reporter compound is selected from a group of compounds consisting of: pigments, dyes, electrochemical active compounds, enzymes, flourophores, chemiluminescent molecules and radionuclides.

It is another object of the present invention to provide the aforementioned sensor, wherein preventative pad consists of at least one compound that can be altered by interacting with the effector.

It is another object of the present invention to provide the aforementioned sensor, wherein the preventative layer is comprised of material that can be digested by an enzyme, the material selected from a group of organic compounds, the group consisting of sugars, hydrogels, peptides, lipids and polymers.

It is another object of the present invention to provide the aforementioned sensor, wherein the absorption pad selectively or non-selectively reacts and/or binds with the reporter compound to generate a signal.

It is another object of the present invention to provide the aforementioned sensor, wherein the signal is identified visually or measured by a detector.

It is another object of the present invention to provide the aforementioned sensor, wherein the detector is based on spectroscopic, photochemical, biochemical, enzymatic, immunochemical, electrical, radiographic and optical means.

It is another object of the present invention to provide the aforementioned sensor, wherein the detector communicates results to a computer system or network.

It is the object of the present invention to provide method for analyzing a sample comprising steps of:
a. obtaining a sample as a solution;
b. obtaining a sensor, the sensor comprising;
   i. at least one reversibly immobilized analyte-effector complex;
   ii. at least one reporter compound;
   iii. at least one preventative layer; and
   iv. at least one absorption pad;
      wherein the reporter compound and the analyte are not bound to each other; wherein the preventative layer is positioned between the analyte and the reporter and the detector; wherein the preventative layer is positioned between the analyte and the reporter and between the absorption layer;
c. loading sample solution; and
d. reading analysis result.

It is another object of the present invention to provide the aforementioned method, wherein the sample layer is constructed from a porous, absorbent and nonreactive membrane.

It is another object of the present invention to provide the aforementioned method, wherein the membrane is selected from a group comprising: cellulose acetate membrane, nitrocellulose membrane, cellulose ester membrane, polysulfone (PS) membrane, polyether sulfone (PES) membrane, polyacrylonitrile (PAN) membrane, polyamide membrane, polyimide membrane, polyethylene and polypropylene (PE and PP) membrane, polytetrafluoroethylene (PTFE) membrane, polyvinylidene fluoride (PVDF) membrane, polyvinylchloride (PVC) membrane and fiberglass paper membrane.

It is another object of the present invention to provide the aforementioned method, wherein the reaction pad contains at least one kind of immobilized analyte-effector complex.

It is another object of the present invention to provide the aforementioned method, wherein the analyte-effector complex is released from the antibody through specific competitive or non-competitive binding of the free analyte from the sample to the anti-body, immobilized to the reaction layer.

It is another object of the present invention to provide the aforementioned method, wherein the reporter compound is characterized by being:
a. incapable of crossing un-affected preventative layer; and
b. capable of crossing affected preventative layer; and
c. specific to the absorption pad or the detector.

It is another object of the present invention to provide the aforementioned method, wherein the reporter compound is selected from a group of compounds consisting of: pigments, dyes, electrochemical active compounds, enzymes, flourophores, chemiluminescent molecules and radionuclides.

It is another object of the present invention to provide the aforementioned method, wherein preventative pad consists of at least one compound that can be altered, by interacting with the effector.

It is another object of the present invention to provide the aforementioned method, wherein the preventative layer is comprised of at least one material that can be digested by an enzyme, the material selected from a group, the group consisting of sugars, hydrogels, peptides, lipids and polymers.

It is another object of the present invention to provide the aforementioned method, wherein the absorption pad selectively or non-selectively reacts and/or binds with the reporter compound to generate a signal.

It is another object of the present invention to provide the aforementioned method, wherein the signal is identified visually or measured by a detector.

It is another object of the present invention to provide the aforementioned method, wherein the detector is based on spectroscopic, photochemical, biochemical, enzymatic, immunochemical, electrical, radiographic and optical means.

It is another object of the present invention to provide the aforementioned method, wherein the detector communicates results to a computer system or network.

### BRIEF DESCRIPTION OF THE FIGURES

FIG. 1—illustrates the schematic structure of the sensor.

FIG. 2—illustrates the schematic activity of the sensor.

FIG. 3—illustrates the schematic activity of the sensor.

FIG. 4—demonstrates the effect of the deposition processes on the uniformity of the gelatin layer

FIG. 5—demonstrates the effect of the gelatin concentration on the solution preventing properties.

FIG. 6—demonstrates that an increase in the gelatin concentration decreases the capability of the enzyme in the solution diffused through the preventative layer

FIG. 7—demonstrates the proof of concept of the present invention

### DETAILED DESCRIPTION OF THE INVENTION

In this application, the term “Analyte” or “analyte of interest” as used herein, refers to the substance to be detected, which may be present in the liquid sample. The analyte can be any substance for which there exists at least one naturally occurring or synthetic specific binding partner. The analyte can include a protein or protein fragment, a polypeptide peptide or peptide fragment, an amino acid, a DNA fragment, a RNA fragment, a small molecule, a bacterium, natural ligands, virus particles (virions), a virus or metabolites of or antibodies to or biomimetic of any of the above substances. The analyte can be a polutent or can serve as a pesticide or a toxin. In some configurations, the identified analytes could be only segments of the origional analyte, cased by fragmentation, desintgration, deterioration, deacy, oxidation etc. Fragmentation can be caused by exposure to the eviroment or as part of the sample preperation procedure.

In this application, the term an “anti-analyte antibody” refers to a protein consisting of one or more polypeptides substantially encoded by immunoglobulin genes or fragments of immunoglobulin genes. This term encompasses polyclonal antibodies, monoclonal antibodies, and fragments thereof, as well as molecules engineered from immunoglobulin gene sequences. The anti-analyte antibody is specific to the analyte of interest. The term an “anti-analyte capture antibody” is an anti-analyte antibody that captures the analyte of interest. Such antibodies are conveniently affixed to a solid phase, such as the membrane of the reaction layer.

In this application, the term “Reporter compound” or “reporter molecule” (or simply “reporter”) as used herein, refers to molecules useful for detecting the presence, intensity or quantity of the analyte due to an interaction between the reporter compound and the absorption layer and/or a detector. Molecules are detectable by spectroscopic, photochemical, biochemical, enzymatic, immunochemical, electrical, radiographic and optical means. Optically detectable molecules can be detectable in the in the ultraviolet, visual or infrared spectrum, including compounds such as dyes or fluorescent labels.

“Reporter compounds” useful in the present invention also include any suitable molecule which may be conjugated to the analyte molecule without compromising the ability of the reporter molecule to be detected or the analyte to be bound to the anti-body.

In preferred embodiments, the reporter is selected from the group consisting of a dye, a radionuclide, an enzyme and combinations thereof.

The dye can be either “small molecule” dye/fluors, or macromolecule dye/fluors (e.g. green fluorescent proteins and all variants thereof). The dye may be a tandem fluorophore conjugate. In various embodiments, the dye may be a fluorescent semiconductor nanocrystal particle, a quantum dot, an electroactive molecule/dye or an upconversion nanocrystal.

In one preferred embodiment, the reporter compound is loaded on to the “reporter layer” or “reporter membrane”. The reporter layer can be formed by binding the reporter in a selective or non-selective manor. In one preferred configuration, the membrane is loaded by saturating the membrane with a reporter compound solution, and subsequently drying the loaded membrane, binding the reporter to the membrane in a non-selective manor. In this configuration, as the sample solution rehydrates the layer, the reporter interacts with liquid/solution and migrates along with the flow, to the “preventative layer”. Alternatively, the reporter molecule can be loaded onto another layer, up-stream from the preventative layer.

The term “Preventative layer”, “preventative pad” or “preventative membrane” refers to a treated porous or semi-porous membrane or solid layer constructed from a material that does not permit the passage of reporter molecules unless it is altered by interacting with the effector. The Preventative layer obstructs passage of the reporter compound due to chemical or physical properties of the reporter compound and the preventative layer. In a preferred configuration, the preventative layer is any biological or chemical substances that can be digested by enzymes (sugars, hydrogels, peptides, proteins, fats, plastic polymers, etc.) such as gelatin.

The term “effector” (alternatively “effector compound” or” effector molecule”) refers to a compound that can be bound to the analyte without affecting its activity. The effector has the ability to interact with the preventative layer, thereby changing its physical or chemical properties in a way that enables passage of reporter compounds. The effector can be an enzyme, a macromolecule or a small molecule. The effector can be organic, inorganic or organometallic in composition. In one preferred embodiment, the effector is an enzyme capable of digesting the preventative layer, opening pores large enough for the reporter compound to pass.

As used herein, the term “membrane” refers to a natural or synthetic/artificial membrane. The terms “synthetic membrane” or “artificial membrane” refer to a man-made membrane that is produced from organic material, such as polymers and liquids, as well as inorganic materials. A wide variety of synthetic membranes are well known in the art. In various embodiments, the membranes of the sample layer, the at least one conjugation layer, the at least one preventative layer and the absorption layer are independently selected from the group consisting of cellulose acetate membrane, nitrocellulose membrane, cellulose ester membrane, polysulfone (PS) membrane, polyether sulfone (PES) membrane, polyacrylonitrile (PAN) membrane, polyamide membrane, polyimide membrane, polyethylene and polypropylene (PE and PP) membrane, polytetrafluoroethylene (PTFE) membrane, polyvinylidene fluoride (PVDF) membrane, polyvinylchloride (PVC) membrane and fiberglass paper membrane.

The term “absorption membrane”, “absorption layer” or “absorption pad” refers to a treated membrane that specifically or non-specifically binds to or reacts with the reporter compound. In various embodiments, the absorption layer further comprises at least one substrate for a reporter molecule (or simply a reporter). Reporter substrate, as used herein, is intended to include any substrate capable of interacting with the reporter. Preferably, the interaction between the reporter and the reporter substrate produces a qualitative or quantitative effect. A “reporter substrate” as used herein is a substrate (or substrates) that can facilitate measurement of either the disappearance of a substrate or the appearance of a product in connection with a catalyzed reaction. Reporter substrates can be free in solution or bound (or “tethered”), for example, to a surface, or to another molecule. A reporter substrate can be labelled by any of a large variety of means including, for example, fluorophores (with or without one or more additional components, such as quenchers), radioactive labels, biotin (e.g. biotinylation) or chemiluminescent labels. In case the reporter is horseradish peroxidase, the substrate is preferably luminol.

In some configurations, the absorption layer is part of a detector. In this configuration the absorption pad facilitates the interaction between the reporter compound and the detector. In one configuration this is performed by containing reporter substrates or by capturing free reporter compounds.

The term “detector’ refers to a devise that enables the measurement of reporter compounds that reach the absorption layer or interact with reporter substrates. The measurement can be of the compound itself or of the interaction with the substrate. This interaction can be measured by spectroscopic, photochemical, biochemical, enzymatic, immunochemical, electrical, radiographic or optical means. The detector is configured to utilize Chemometrics to analyze the signal (or signals) generated by the reporter-absorption interaction to detect and measure the amount of analyte present in the sample.

The object of this invention is a sensor for the on-site, real-time, fast and simple analyte detection.

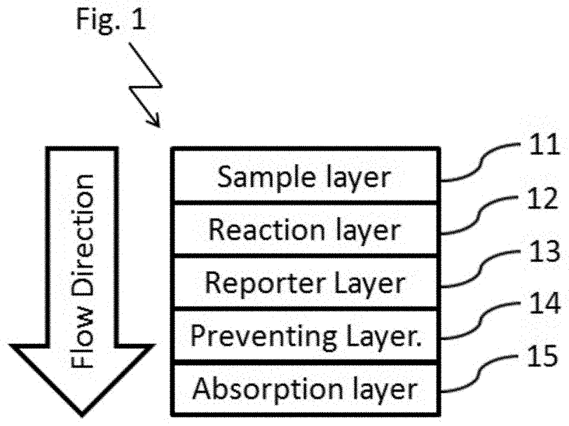

Reference is made to FIG. 1, describing one non limiting embodiment of the invention, a liquid sample collected and loaded on the sample layer (11). The sample solution hydrates the layers and the sample solution flows through to the reaction layer (12). The analyte in the sample binds to the anti-analyte antibody, dislocating the bound analyte-effector complex. The analyte-effector complex passes through the reporter layer to the preventative layer (13). The effector interacts with the preventative layer (13), affecting the physical and/or chemical properties of the preventative layer. This change enables the reporter compounds to traverse the preventative layer and reach the absorption layer (14).

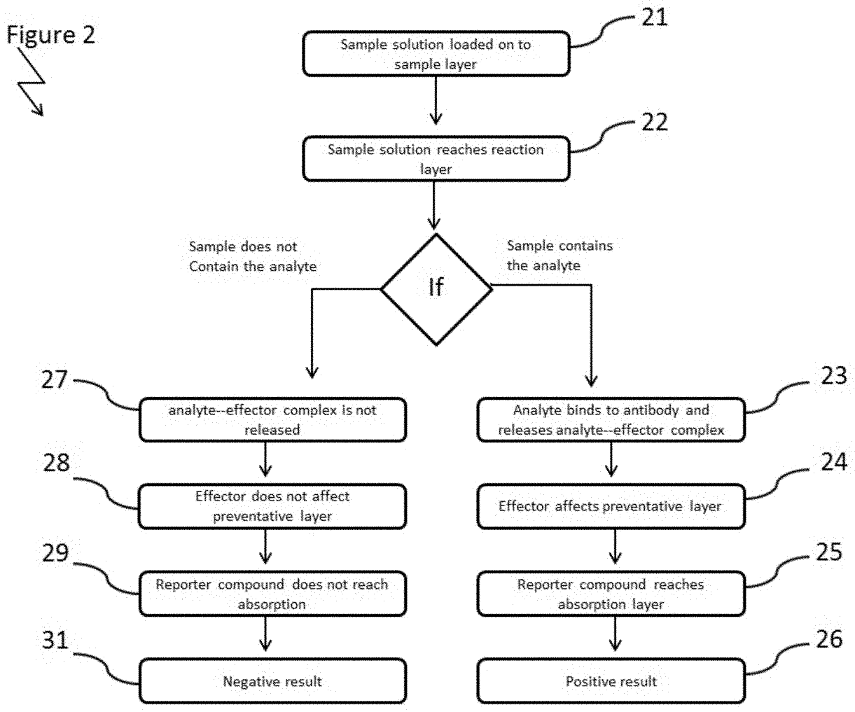

Reference is made to FIG. 2, describing one non limiting embodiment of the invention, a liquid sample collected and loaded on the sample layer 21. The sample solution hydrates the layers and the sample solution flows through to the reaction layer 22. If the sample solution contains the analyte, then the analyte in the sample binds to the anti-analyte antibody, dislocating the bound analyte-effector complex. The analyte-effector complex then passes through the reporter layer to the preventative layer 23. The effector interacts with the preventative layer 23, affecting the physical and/or chemical properties of the preventative layer. This change enables the reporter compounds to traverse the preventative layer 24 and reach the absorption layer 25. The sensor will then return a positive result 26.

If the sample does not contain the analyte, then the analyte-effector complex remains bound 27 and cannot affect the physical and/or chemical properties of the preventative layer 28. The reporter compound will not be able to cross the preventative layer 29 and the sensor will return a negative result 30.

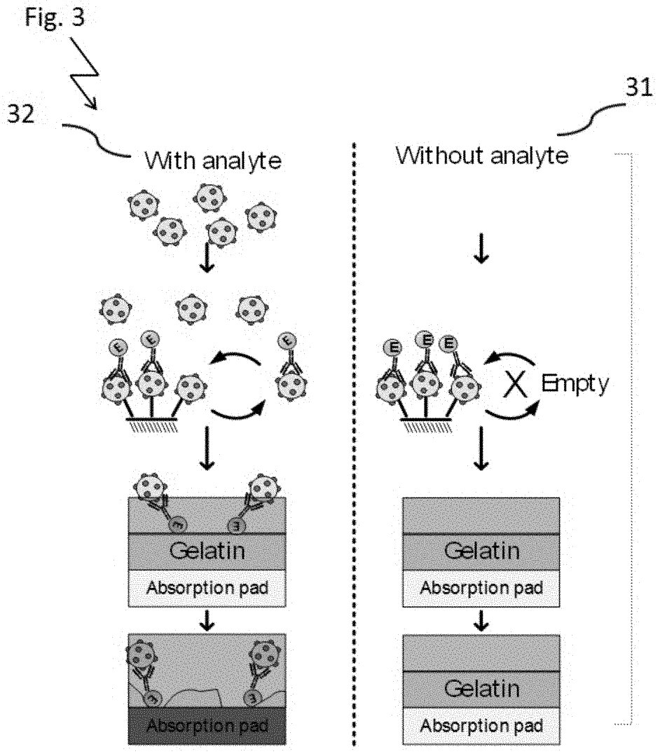

Reference is made to FIG. 3, describing the current invention in two instances:
31 A sample containing the analyte->where the preventative layer interacts with the affector->enabling the reporter compound to reach the absorption layer.
32 A sample not containing the analyte->preventative layer is not affected.

Reporter compounds that traverse the preventative layer interact with the absorption layer to generate a signal. This interaction can be specific, such as binding to a substrate, or non-specific, such as the accumulation of dyes. This interaction generates a signal, such as the generation of a color due to the accumulation of dyes. In some configurations the signal is then measured by the detector. the sensor can be configures to detect the presence of more than one analyte in a single sample by using one reaction layer loaded with multiple anti-analyte capture anti-body's or by using multiple reaction layers, each layer corresponding to a different anti-analyte capture antibody. The detector and absorption layer can be configured to detect the presence of more than one reporter compound, enabling the system to detect the presents of multiple analytes in a single sample. In this configuration, the signal generated by each reporter must be distinctive and must not impede the detectors ability to detect signals generated by other reporter compounds. In this configuration the detector can use chemometrics to detect the level of various analytes in the sample.

In another non limiting embodiment of the invention the sensor is constricted of a number of layers, each layer placed on each other affording to the flow

In another non limiting embodiment of the invention, the sensor is constructed as a single strip. In this embodiment the layers are arraigned end-to-end and are regions of one strip

In this approach, an absorption cellulose membrane served as solid bedding support, onto which different assay components are immobilized onto the various layers.

### Example 1

Herein is described a sensor for detecting allergens in food. In this example the membranes are paper (cellulose), the preventative layer is constructed from gelatin, the reporter compound is a dye and the effector is pepsin. In the first step, the liquid sample is collected and deposited on the sample pad. The sample traverses through a sample pad until it reaches the reaction pad. The reaction pad contains immobilized allergen-pepsin complex (anti-analyte-enzyme complex) bound to an allergen antibody. The free allergen in the sample binds to the anti-analyte antibody capture complex, releasing the analyte-pepsin complex. The complex passes through and rehydrating the color layer and reaches the gelatin preventative layer. The pepsin digests the gelatin, creating pores (one pore for each freed complex). The dyes molecules pass through the pores in the gelatin and reach the absorption pad, coloring the absorption layer.

In this configuration the color indicates the presence of an allergen in the food, alerting to a possible health hazard.

### Example 2

Herein is described a sensor for detecting waterborne pathogens. In this example the membranes are paper (cellulose), the preventative layer is constructed from gelatin, the reporter layer contains red dye and the effector is pepsin. In this configuration the sensor contains 7 reaction layers, each one specific for a different pathogen:
Cryptosporidium
Giardia
Shigella
E. Coli 0157:H7
Legionella
Campylobacter
Salmonella

Each reaction layer contains a specific immobilized pathogen-pepsin complex (anti-analyte-enzyme complex) bound to a pathogen anti-body. Each pathogen-pepsin complex is additionally linked to an additional reporter compound, creating a pathogen-pepsin-reporter complex. Each additional reporter compound is a different florescent compound, each florescent compound emitting light at a distinctive spectrum.

In the first step, the liquid sample is collected and deposited on the sample pad. The sample traverses through a sample pad until it reaches the specific reaction pad. The specific reaction pad contains immobilized pathogen-pepsin-reporter complex bound to the pathogen antibody. The free pathogen in the sample binds to the anti-analyte antibody capture complex, releasing the pathogen-pepsin-reporter complex. The complex passes through and rehydrating the color layer and reaches the gelatine preventative layer. The pepsin than digests the gelatin, creating pores (one pore for each freed complex). The dyes molecules pass through the pores in the gelatin and reach the absorption pad, coloring the absorption pad. The absorption layer is then loaded into a spectroscopic detector can then be used to identify the specific pathogen in the samples.

In this configuration a colored layer warns about a possible water contamination and the use of a detector detects the specific pathogens present in the water source.

### Example 3

Herein is described a method for validating cleaning in place of reactors. In this example the membranes are paper (cellulose), the preventative layer is constructed from gelatin, the reporter compound is a dye and the effector is pepsin. In this example the reported compound is dissolved in the sample collection solution.

In the first step, a dose of the sample collection solution of deposited on the surface of the reactor. The sample collection solution comprises a suitable solvent and the dye (the reported compound). The sample layer is dunked in the solution that is on the reactor surface. The sample traverses through a sample pad until it reaches the reaction pad. The reaction pad contains immobilized analyte-pepsin complex (anti-analyte-enzyme complex) bound to an anti-analyte antibody. The free analyte in the sample binds to the anti-analyte antibody capture complex, releasing the analyte-pepsin complex. The complex reaches the gelatin preventative layer. The pepsin digests the gelatin, creating pores (one pore for each freed complex). The dyes molecules pass through the pores in the gelatin and reach the absorption pad, coloring the absorption layer.

In this configuration the color indicates that the reactor has not been sufficiently cleaned.

### Example 4

Herein is demonstrated the bio-physical effects of the method.

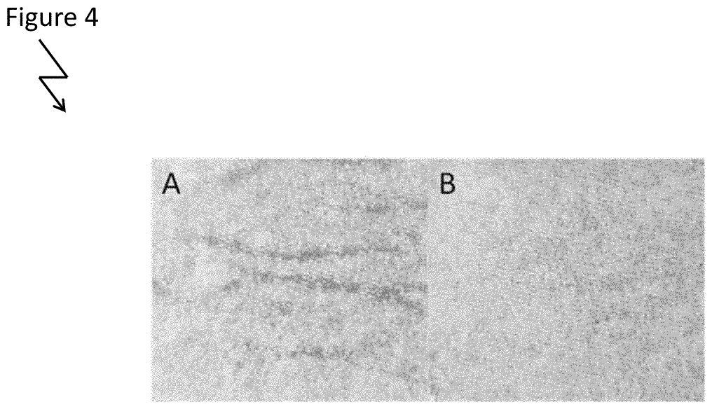

Reference is made to FIG. 4, demonstrating the effect of the deposition processes on the uniformity of the gelatin layer, so as to determine the constant diffusion time gelatin layer supposed to be uniform. To determinate effect of the drying protocol on the uniformity of the gelatin layer formation, two different drying modes were tested:
In the first (FIG. 4A), 250 μl of 5% (w/v) gelatin solution was placed above the 4×4 cm Kim wipes paper and drying on the flat surface at room temperature.
In the second, 4×4 cm Kim wipes paper was dipped in 1 mL 5% (w/v) gelatin solution, squeezed (to remove liquid excesses) and dried in the air stretched out.
Uniformity of the gelatin layer is a critical issue in the sensor development procedure, as a uniform layer not only will allow determination constant passing time through this stopping layer in the sensor, but also will be disassembled by the same enzyme concentrations during evaluation.

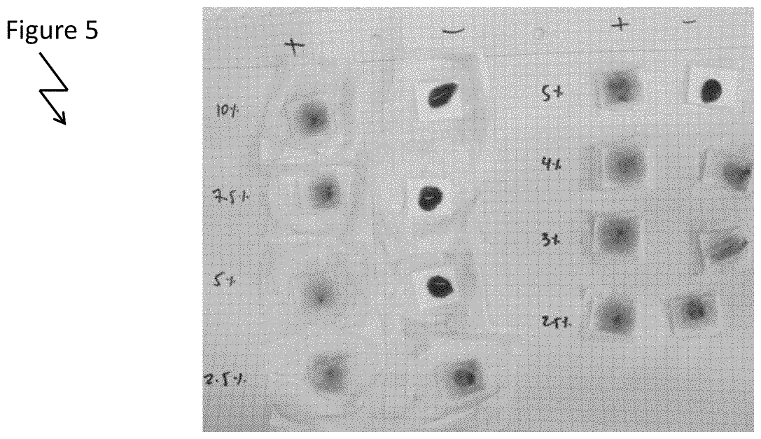

Reference is made to FIG. 5, demonstrating the effect of gelatin concentration on the ability of the preventative layer to prevent/stop/regulate the passage of the solution. Prevention layers with different gelatin concentrations were created as described in the previous section (for FIG. 4). The prevention layers were then placed above the absorption layer and 25 μl of colored solution with (+) and without (−) enzyme bromelain (250 units/mL) were placed above it. Bromelain is enzyme from pineapples that degrades gelatin, thus testing the stopping capabilities of the gelatin layers (with clear solution) and also determining effect of various gelatin concentrations on enzymatic reaction of the positive (+) solution (the solution containing the enzyme). FIG. 5 demonstrates that all solutions containing the Bromelain enzyme diffused through preventing layer, regardless of the gelatin concentration. This demonstrates the ability of the Bromelain enzyme to penetrate the preventative layer at any used gelatin concentrations. In the enzyme negative samples (tested with clear water), only layers with 5% gelatin (w/v) and higher showed the ability to stop the solution. This indicates the specificity of the diffusion/degradation mechanisms, where systems with a preventative layer containing at least 5% (w/v) gelatin will allow the passage of enzyme containing solutions while stopping negative samples and will pass solutions.

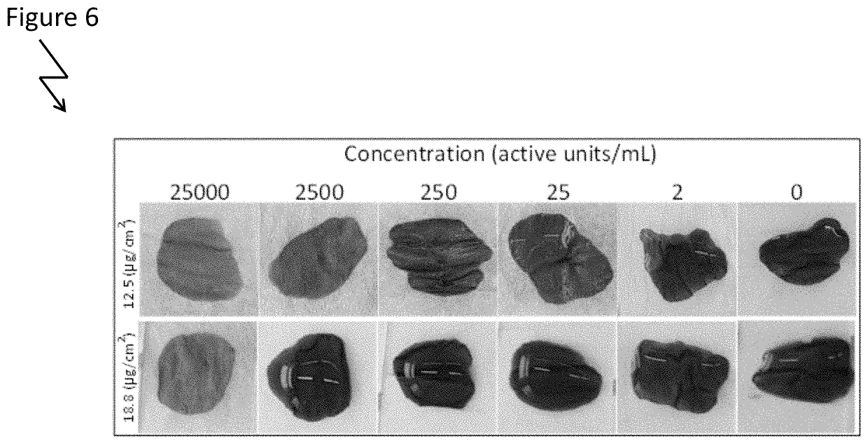

Reference is made to FIG. 6, showing the determining effect of the gelatin concentration on its preventing/stopping properties. For this step, preventative layers with 5% (w/v) gelatin concentration where generated as described in the FIG. 4 and placed above absorption pad. To test enzyme passing capability, different bromelain concentrations were prepared in colored solution and placed above preventative layers. As the enzyme degrades the gelatin in the preventative layer, the colored solution passes through to the absorption layer and colored it. This step not will show minimum enzyme concentration that may pass stopping layer and effect of gelatin concentrations on these passing capabilities.

FIG. 6 demonstrates that an increase in the gelatin concentration in the preventative layer lowers the ability of the Bromelain enzyme in the solution to enable diffuse of the colored solution through preventative layer. While both tested concentrations prevent false-negative results, a preventative layer containing 12.5 μg/mL gelatin were enough to prevent solutions containing two active units to diffuse or pass through preventative layer. The negative samples show that a preventative layer containing 5% (w/v) gelatin prevents the negative solution (without enzyme) diffusion through it, as a positive solution (containing the Bromelain enzyme) can disassemble the preventative layer and enable the passage of the solution, with as low an enzyme concentration as possible.

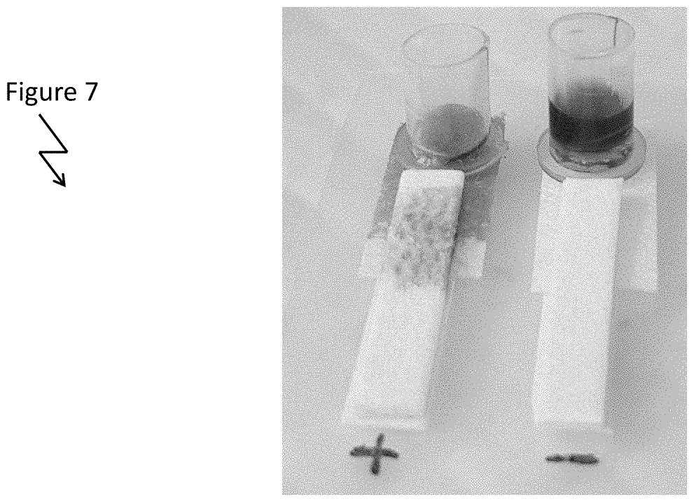

FIG. 7 further demonstrates the potential of the present invention to generate a positive result when in the present of a solution containing the Bromelain enzyme (+).

1.-48. (canceled)

49. A sensor for the rapid onsite identification of analytes comprising:
a. at least one reversibly immobilized analyte-effector complex;
b. at least one reporter compound;
c. at least one preventative layer; and
d. at least one absorption layer;
wherein at least one of said reporter compounds and said analyte are not bound to each other; and said preventative layer is positioned between said analyte and said reporter and between said absorption layer and is configures as a specific analyte signaling channel.

50. The sensor of claim 49, wherein said analytic-effector complex is characterized by at least one of the following:
a. comprising:
i. at least one analyte; and
ii. at least one effector, said effector specific to composition of said preventative pad;
wherein said analyte and effector are reversibly or irreversibly connected;
b. said analyte-effector complex is immobilized by being reversibly bound to an anti-analyte antibody, said anti-analyte antibody is specific for said analyte.

51. The sensor of claim 50, wherein said effector is an enzyme.

52. The sensor of claim 50, wherein said analyte-effector complex is released from said antibody through specific competitive or non-competitive binding of said free analyte from said sample to said anti-body, immobilized to said reaction layer.

53. The sensor of claim 49, wherein said reporter compound is characterized by being:
a. incapable of crossing un-affected preventative layer; and
b. capable of crossing affected preventative layer; and
c. specific to said absorption pad or said detector.

54. The sensor of claim 53, wherein said reporter compound is selected from a group of compounds consisting of: pigments, dyes, electrochemical active compounds, enzymes, flourophores, chemiluminescent molecules and radionuclides.

55. The sensor of claim 49, wherein preventative pad consists of at least one compound that can be altered, by interacting with said effector, said material selected from a group consisting of sugars, hydrogels, peptides, lipids and polymers.

56. The sensor of claim 49, wherein said absorption pad selectively or non-selectively reacts and/or binds with said reporter compound to generate a signal.

57. The sensor of claim 56, wherein said signal is identified visually or measured by a detector.

58. The sensor of claim 57, wherein detector is characterized by at least one of the following:
a. based on spectroscopic, photochemical, biochemical, enzymatic, immunochemical, electrical, radiographic and optical means;
b. communicating said results to a computer system or network;

59. A method for analyzing a sample comprising steps of:
a. obtaining a sample as a solution;
b. obtaining a sensor, said sensor comprising;
i. at least one reversibly immobilized analyte-effector complex;
ii. at least one reporter compound;
iii. at least one preventative layer; and
iv. at least one absorption layer;
wherein at least of said reporter compounds and said analyte are not bound to each other; wherein said preventative layer is positioned between said analyte and said reporter and between said absorption;
c. loading sample solution; and
d. reading analysis result.

60. The method of claim 59, wherein said reaction pad contains at least one kind of immobilized analyte-effector complex.

61. The sensor of claim 60, wherein said analyte-effector complex is released from said antibody through specific competitive or non-competitive binding of said analyte from said sample to the anti-body, immobilized to said reaction layer.

62. The method of claim 59, wherein said reporter compound is characterized by being:
a. incapable of crossing un-affected preventative layer; and
b. capable of crossing affected preventative layer; and
c. specific to said absorption pad or said detector.

63. The method of claim 62, wherein said reporter compound is selected from a group of compounds consisting of pigments, dyes, electrochemical active compounds, enzymes, flourophores, chemiluminescent molecules and radionuclides.

64. The method of claim 63, wherein preventative pad consists of at least one compound that can be altered by interacting with said effector, said material selected from a group consisting of sugars, hydrogels, peptides, lipids and polymers.

65. The method of claim 59, wherein said absorption pad selectively or non-selectively reacts and/or binds with said reporter compound to generate a said analysis result.

66. The method of claim 65, wherein said analysis result is a signal, said signal is identified visually or measured by a detector.

67. The method of claim 66, wherein detector is based on spectroscopic, photochemical, biochemical, enzymatic, immunochemical, electrical, radiographic and optical means.

68. The method of claim 66, wherein detector communicates said results to a computer system or network.
